(19) United States
(12) Patent Application Publication
(10) Pub. No.: US 2024/0132813 A1
(43) Pub. Date: Apr. 25, 2024

(54) MONITORING AND CONTROLLING YEAST PROPAGATION

(71) Applicant: Endress+Hauser SE+Co. KG, Maulburg (DE)

(72) Inventors: Julia Schon, Aldingen (DE); Julia Rosenheim, Wehr (DE); Tobias Brengartner, Emmendingen (DE); Sergey Lopatin, Lörrach (DE)

(21) Appl. No.: 18/546,542

(22) PCT Filed: Jan. 27, 2022
(86) PCT No.: PCT/EP2022/051857

§ 371 Date: Aug. 15, 2023

(30) Foreign Application Priority Data
Feb. 18, 2021 (DE) 10 2021 103 928.2

(51) Int. Cl. C12C 11/02; G01N 33/14

(52) U.S. Cl. CPC C12C 11/02; G01N 33/146

(57) ABSTRACT

A computer-implemented method for controlling and/or monitoring yeast propagation includes providing a mixture of yeast cells and wort, determining an original gravity of the wort or of the mixture and a temperature of the mixture at a start time, and calculating a theoretical end time for the yeast propagation on the basis of a logistic propagation model for the yeast propagation, on the basis of the original gravity and on the basis of the temperature at the start time.

The present invention relates to a method, in particular to a computer-implemented method, for controlling and/or monitoring yeast propagation—for example, in an industrial-scale plant. In particular, the invention relates to yeast propagation in a brewery.

The production of beer comprises several complex method steps, such as the malting of grain, mashing, lautering, wort boiling, and fermentation. For reliable monitoring and/or control and optimization of such complex processes, simultaneous knowledge of a wide variety of process variables and the underlying relationships in each case is required. For example, DE102019110821A1 discloses a method for determining and/or monitoring a concentration of maltodextrin and/or maltose on the basis of the density and the sonic velocity in a mashing process. As a result, the duration required for saccharification of the pitched mash can be precisely determined.

Wort fermentation refers to the pitching or inoculation of hopped pitching wort or wort with a yeast. Yeast propagation is usually carried out in a so-called batch operation, in which a fresh yeast culture is prepared in the wort in each batch and used completely for the subsequent fermentation. In this context, yeast propagation is understood to mean the increase in the biomass and cell number of the respective yeast population, which occurs in several different growth phases. Ideally, the result should be a yeast with the highest possible yeast vitality, i.e., high fermentability and viability. In order to ensure this, various measurements can be taken during the fermentation process with or without sampling, on the basis of which the process of yeast propagation can be monitored. However, a uniform or common procedure with regard to monitoring and/or controlling yeast propagation is not available. Such a procedure is also difficult to implement for such a multivariant biological system with a complex growth behavior.

Therefore, the object of the present invention is to improve the control and/or monitoring of yeast propagation.

This object is achieved by a method, in particular a computer-implemented method, for controlling and/or monitoring yeast propagation, comprising the following method steps:
providing a mixture of yeast cells and wort,
determining an original gravity of the wort or of the mixture and a temperature of the mixture at a start time, and
calculating a theoretical end time for the yeast propagation on the basis of a logistic propagation model for the yeast propagation, on the basis of the original gravity, and on the basis of the temperature at the start time.

The original gravity in degrees Plato (° P) refers to the content of dissolved ingredients from malt and hops in the wort before the start of fermentation and at a start time of yeast propagation. During the yeast propagation, this value decreases as a result of yeast metabolism. The content of dissolved ingredients from malt and hops after completion of the yeast propagation is called the residual extract content.

By taking into consideration a logistic propagation model, it is possible to accurately predict a time duration for the process of yeast propagation. The invention is based upon the knowledge that, from among a plurality of possible influencing factors, the original gravity and temperature at the start time are decisive for yeast propagation. Monitoring and/or control of yeast propagation can thus be implemented in a simple manner; in particular, a duration for the yeast propagation can be optimized by predicting the end time.

One embodiment of the method includes determining a free amino nitrogen (FAN) content of the wort and taking it into account for the calculation of the theoretical end time on the basis of the propagation model. The FAN content plays a role in particular for the metabolic activities of the yeast, for the yeast growth, and the physiological state of the yeast. The FAN content can, for example, be determined regularly by sampling—for example, during the performance of process checks in the brewery.

A further embodiment of the method includes determining or measuring the temperature of the mixture during the yeast propagation. Temperature is a dominant factor in the growth rate of yeast cells, and thus significantly influences the rate of yeast propagation. When a temperature change is detected, the prediction for the end time of the yeast propagation can be suitably corrected, for example.

In one embodiment, an actual value for the extract content of the mixture is determined or measured during the yeast propagation. The extract content can be used, for example, to monitor the nutrient supply to the yeast during the process of yeast propagation.

In addition, it is advantageous if an alcohol content, in particular an ethanol content, of the mixture is determined or measured during the yeast propagation. During yeast propagation, there is an accumulation of extracellular ethanol, which also influences the growth rate of the yeast.

It is further advantageous if a dissolved oxygen content of the mixture is determined or measured during the yeast propagation. This variable, in turn, can be used to verify and/or monitor the presence of saturation of the wort.

One embodiment of the method includes using a model for an extract content in the mixture as the propagation model, wherein the extract content is determined on the basis of the original gravity at the start time, a target value for the residual extract content, and taking into account a substrate uptake rate, a temperature rate, and a duration for a lag phase during the yeast propagation.

Specifying a target value for the residual extract content serves to ensure sufficient nutrient supply and vitality of the yeast until the end time of the yeast propagation.

Using the substrate uptake rate, the specific growth kinetics of the yeast propagation can be included in the propagation model. For the yeast propagation, all substrates of the yeast are fed in at the beginning of the process. Thus, the yeast cells generally grow until the substrates are exhausted.

The temperature rate also significantly influences the speed of yeast propagation and is therefore included in the determination of the growth rate within the propagation model.

Finally, the lag phase concerns a delay phase at the beginning of yeast propagation, which occurs in particular when the yeast culture is over-inoculated into the wort. During this phase, the yeast cells are biochemically active, but they do not divide. Thus, including the duration of the lag phase to accurately predict an end time for the yeast propagation is also advantageous.

One embodiment includes determining a reference value for the extract content on the basis of the propagation model. The reference value can, for example, be determined continuously or at predefinable times during the yeast propagation.

A further embodiment comprises comparing the actual value for the extract content with the reference value for the extract content, and wherein, in the event that a deviation between the actual value and reference value exceeds a predefinable limit value, at least one influencing quantity for the yeast propagation is varied as a function of the deviation. Ideally, the time at which the actual value is determined is taken into account for determining the reference value for the extract content, or both values are determined for the same time. The comparison thus enables continuous monitoring and/or control of the process.

In this connection, it is advantageous if the at least one influencing quantity is the temperature, the end time of the yeast propagation, or the concentration of dissolved oxygen. Thus, during the yeast propagation, a temperature or a supply of oxygen can be suitably adjusted, and in particular regulated or controlled. Additionally or alternatively, the end time for the yeast propagation predicted on the basis of the propagation model can be adapted or changed on the basis of the respective conditions.

For example, it is conceivable to adjust a temperature of the mixture during the yeast propagation on the basis of the deviation between the actual value and the reference value of the extract content, and in particular to regulate or control it in this way.

Likewise, one embodiment includes adjusting, in particular regulating or controlling, aeration of the mixture during the yeast propagation, on the basis of the deviation between the actual value and the reference value of the extract content and the dissolved oxygen content.

One embodiment of the method includes determining a pitching concentration of the yeast at the start time.

In this connection, it is advantageous if a biomass concentration of the yeast is determined on the basis of the propagation model, the pitching concentration, and a target value for the yeast concentration, which is related to the target value for the residual extract content.

In this connection, it is also advantageous if a viable yeast cell concentration during the propagation is determined on the basis of the biomass concentration. This advantageously allows monitoring of the cell concentration without direct measurement.

Finally, a further embodiment includes determining a pH value of the mixture. This in turn serves to monitor the microbiological safety of the process.

The various influencing quantities which play a role in yeast propagation, such as, for example, the extract content, the FAN content, the temperature, the alcohol or ethanol content, the dissolved oxygen content, or the pH value, can each be determined at specific, predefinable times or continuously. It is conceivable both to determine individual influencing quantities by means of sampling and subsequent analysis and to determine them directly in the process. For direct measurement, suitable sensors for determining the respective influencing quantity can be attached to or in a container which is used to perform the yeast propagation. These can be thermometers, oxygen sensors, and/or pH sensors. To determine density and/or viscosity and with influencing quantities, dependent upon these variables, such as alcohol content, reference is made in this connection, for example, to the devices and methods described in DE102018127526A1, DE102016120326A1, DE102016111134, or DE102015112421A1, or also DE102014119061A1.

On the basis of the propagation model, it is possible to predict an ideal profile for the yeast propagation under given conditions with respect to the time duration and the desired cell number of living cells, and to control it appropriately by determining at least one influencing quantity at the same time.

The object underlying the invention is further achieved by a computer program for controlling and/or monitoring yeast propagation, having computer-readable program code elements which, when executed on a computer, cause the computer to execute the method according to the invention in accordance with at least one of the embodiments described.

The object underlying the invention is likewise achieved by a computer program product having a computer program according to the invention and at least one computer-readable medium on which at least the computer program is stored.

The method can be carried out both on a computing unit and a computer at the site where the yeast propagation is performed—for example, in the brewery. However, it can also be carried out by means of an external computing unit, provided that necessary measurement data are transmitted to this computing unit. For example, the method may be implemented by a cloud application. In this case, the measuring devices are designed to determine values of the aforementioned influencing quantities, such as a thermometer or an oxygen sensor, preferably with means for transmitting measured values to the cloud.

The invention and its advantageous embodiments are explained in further detail with reference to the following figures. In the figures:

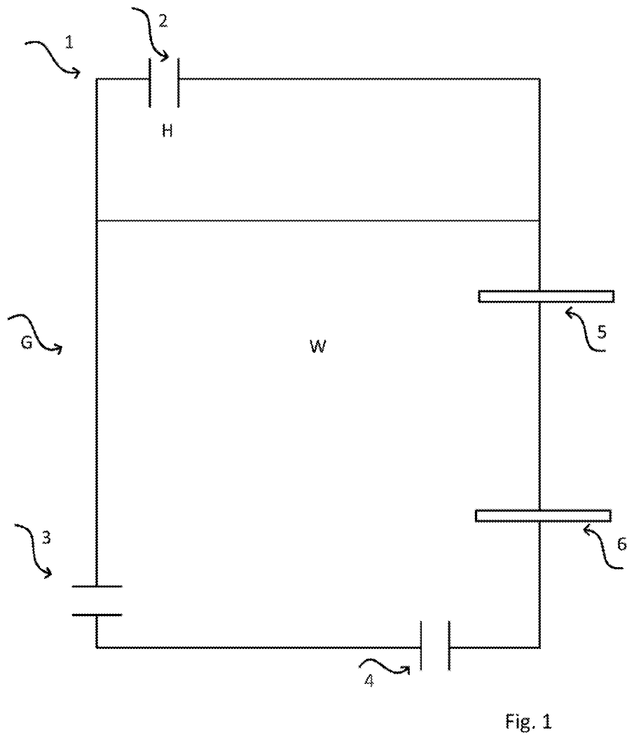

FIG. 1 shows an example of a container for performing yeast propagation; and

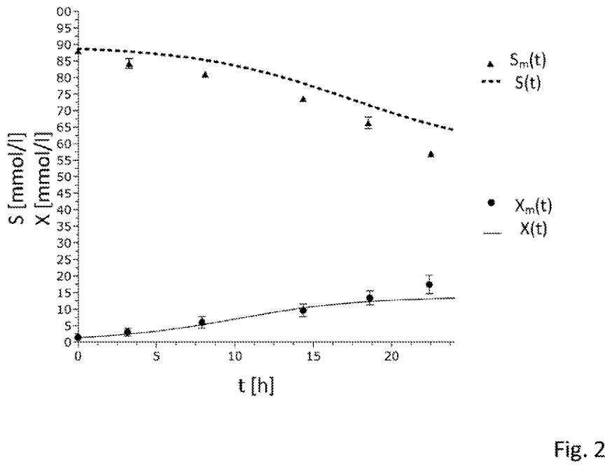

FIG. 2 shows the residual extract and the biomass concentration, in each case in the form of measured curves and reference curves calculated by means of the propagation model. In the figures, identical elements are provided with the same reference signs.

FIG. 1 shows a schematic and exemplary illustration of a container 1 for yeast propagation in a brewery. The container 1 comprises an inlet 2 for pitching the hopped wort W with yeast H. Advantageously, the pitching concentration of the yeast H, the temperature T inside the container 1, and, optionally, the FAN content of the yeast H are determined at the beginning of the yeast propagation. Subsequently, the process of yeast propagation begins for the mixture G.

The container 1 further comprises an outlet 3 for removal and a device 4 for aerating the container 1.

For monitoring and/or control purposes, an oxygen sensor 5 and a thermometer 6 are further incorporated into the tank in the example shown here. A heating/cooling device [not illustrated] may further be provided for adjusting the temperature T in the container 1 during the yeast propagation.

The present invention provides a way to monitor and/or control yeast propagation. A growth process under controlled conditions and harvesting of the yeast at the right time, i.e., when the cell number and vitality of the yeast are as high as possible, are made possible.

Numerous descriptions of various influencing quantities of yeast propagation, as well as models for describing the process of yeast propagation, are already known from the prior art, such as from the dissertation, “Mathematically Based Management of Saccharomyces sp. Batch Propagation and Fermentations,” by T. Kurz (2002) at the Technical University of Munich, or in “Modeling of the Bacterial Growth Curve” by M. H. Zwietering et al. in Applied and Environmental Microbiology, pp.1875-1881, 1990. However, since yeast propagation is a complex, multivariant problem, the available models make use of a plurality of variables or different influencing quantities and are of limited suitability for practical control and/or monitoring of yeast propagation. Moreover, many of the influencing quantities are difficult to obtain directly, in particular continuously, during yeast propagation. The present invention thus relates to a simplification of the known models and descriptions, which allows targeted control and/or monitoring of yeast propagation. An essential finding underlying the invention relates to targeted selection of significant influencing quantities in the creation of the propagation model.

The starting point for modeling yeast propagation is a logistic propagation model as described in (Speers, et al., 2003), which describes the extract decrease S as a function of time t, a substrate uptake rate μₛ, an extract difference ΔS=S₁−S₂ between the original gravity S₁ and a target value for the residual extract content S₂, and a duration for the lag phase λ:

$$S ( t ) = {S}_{2} - \frac{Δ S}{1 + {e}^{( {μ}_{s} ( λ - t ) )}}$$

The determination of the substrate uptake rate μₓ is based upon the application of Monod kinetics to account for a dependence of the growth rate R upon a limiting substrate concentration during the growth process due to nutrient exhaustion, accumulation of toxic metabolic products, and the ion balance. As described in the article, “Growth of Saccharomyces cerevisiae is controlled by its limited respiratory capacity: formulation and verification of a hypothesis,” by B. Sonnenleitnert and O. Käppeli, published in Biotechnology and Bioengineering, Vol. XXVIII, pp. 927-937, John Wiley & Sons, Inc., 1986, the substrate uptake rate μₓ is given by:

$${μ}_{s} = {μ}_{s max} \cdot min ( \frac{Z}{Z + {K}_{Z}} , \frac{N}{N + {K}_{n}} ) \cdot \frac{{K}_{E}}{{K}_{E} + E}$$

Here, Z describes the sugar concentration, N the nitrogen concentration, E the ethanol content, μ_{s,max} the maximum specific growth rate, and Kₛ the half-maximum concentration of the substrates.

The dependence upon temperature T in the growth of microorganisms as the dominant factor influencing the growth process is based upon an extended form of the Bělehrádek model for the temperature rate r, as described in “Model for Bacterial Culture Growth Rate Throughout the Entire Biokinetic Temperature Range” by D. A. Ratowsky et al., published in Journal of Bacteriology, pp. 1222-1226, 1983:

\sqrt{r}=α*(T−Tₘₐₓ)*{1−e^{[b*(T-T}^{ₘₐₓ}^{)]}}.

Here, Tₘᵢₙ and Tₘₐₓ describe a minimum and maximum growth temperature, respectively, and a, b empirical parameters. Based upon this temperature rate, the duration of the lag phase λ is given as a function of the temperature T:

λ=(α(T−Tₘᵢₙ)·{1−e^{[b·(T-T}^{ₘₐₓ}^{)]}})⁻²,

as proposed in “Modeling of Bacterial Growth with Shifts in Temperature” by M. H. Zwietering et al., Applied and Environmental Microbiology, pp. 204-213, 1994.

The extract content S can thus be determined as a function of time t on the basis of the following propagation model S(t):

$$S ( t ) = {S}_{2} - \frac{( Δ S )}{1 + {e}^{- 〈 {μ}_{s max} \cdot min ( \frac{S}{S + {K}_{S}} , \frac{N}{N + {K}_{n}} ) \cdot {( a \cdot ( T - {T}_{min} ) \cdot \{ 1 - {e}^{[ b \cdot ( T - {T}_{max} ) ]} \} )}^{2} 〉 ( t - {( a \cdot ( T - {T}_{min} ) \cdot \{ 1 - {e}^{[ b \cdot ( T - {T}_{max} ) ]} \} )}^{- 2} + c )}}$$

Here, c represents a further free parameter.

On the basis of the propagation model S(t) according to the invention, it is possible to determine a theoretical end time t_{end} for the yeast propagation on the basis of the original gravity E₁ and the temperature T at a start time tₛₜₐᵣₜ. dS(t)

The extract decrease dS(t)/dt is related to the biomass increase dX(t)/dt, i.e., to the biomass concentration X(t) as a function of time t. The extract decrease dS(t)/dt corresponds to the nutrient uptake of the yeast, and therefore the biomass concentration X increases proportionally with the decrease in 25 extract S. The proportionality factor Y is referred to as biomass yield and represents the amount of biomass per substrate. Thus, the rate of biomass increase dX(t)/dt or the biomass growth rate μₓ is obtained on the basis of the substrate uptake rate μₛ as:

μₓ=Y·μₛ

It is thus possible to calculate the increase in biomass ΔX from the extract difference ΔE. The biomass concentration X or the yeast concentration can be determined from the decrease in the extract S without the need to perform another direct measurement. Cell concentration monitoring is thus also possible in this way.

In FIG. 2, the residual extract S and the biomass concentration X are each shown as a function of time tin the form of measured values (Sₘ(t) and Xₘ(t), respectively) determined in a test measurement, and of reference curves (dashed and solid line) calculated by means of the propagation model S(t) and X(t), respectively. In this context, for the propagation model S(t), an extract difference ΔS=4%, while the measured extract difference ΔS was slightly larger. The model reflects the process of yeast propagation in a satisfactory manner.

### REFERENCE SIGNS

1 Container
2 Inlet
3 Outlet
4 Aeration device
5 Oxygen sensor
6 Thermometer
W Wort
H Yeast
T Temperature
FAN FAN content
G Mixture of wort and yeast
S Extract decrease
t Time
ΔS Extract difference
S₁ Original gravity
S₂ Target value for residual extract content
λ Duration of lag phase
μₓ Substrate uptake rate
R Growth rate
Z Sugar concentration
N Nitrogen concentration
E Ethanol content
μ_{s,max} Maximum specific growth rate
Kₛ Half-maximum concentration of substrates
Tₘᵢₙ Minimum growth temperature
Tₘₐₓ Maximum growth temperature
a,b Empirical parameters
S(t) Propagation model
tₛₜₐᵣₜStart time
t_{end} End time
X(t) Biomass concentration
Y Proportionality factor, biomass yield
μₓ Biomass growth rate
ΔX Increase in biomass
Sₘ(t) Measured values for extract content
Xₘ(t) Measured values for biomass concentration

1-15. (canceled)

16. A computer-implemented method for controlling and/or monitoring yeast propagation, comprising:
providing a mixture of yeast cells and wort;
determining an original gravity of the wort or of the mixture and a temperature of the mixture at a start time; and
calculating a theoretical end time for the yeast propagation on the basis of a logistic propagation model for the yeast propagation, on the basis of the original gravity, and on the basis of the temperature at the start time.

17. The method according to claim 16, further comprising:
determining a free amino nitrogen (FAN) content of the wort;
wherein the FAN is taken into account for the calculation of the theoretical end time on the basis of the propagation model.

18. The method according to claim 16,
wherein the temperature of the mixture is determined during the yeast propagation.

19. The method according o claim 16, further comprising:
determining an actual value for an extract content of the mixture during the yeast propagation.

20. The method according to claim 16, further comprising:
determining an alcohol content of the mixture during the yeast propagation.

21. The method according to claim 16, further comprising:
determining a dissolved oxygen content of the mixture during the yeast propagation.

22. The method according to claim 16,
wherein a model for an extract content in the mixture is used as the propagation model,
wherein the extract content is determined on the basis of the original gravity at the start time, a target value for a residual extract content, a substrate uptake rate, a temperature rate, and a duration for a lag phase during the yeast propagation.

23. The method according to claim 22, further comprising:
determining a reference value for the extract content on the basis of the propagation model.

24. The method according to claim 23, further comprising:
comparing an actual value for the extract content with the reference value for the extract content; and
when a deviation between the actual value and the reference value exceeds a predefinable limit value, varying as a function of the deviation at least one influencing quantity for the yeast propagation.

25. The method according to claim 24,
wherein the at least one influencing quantity is the temperature, the end time of the yeast propagation, or a concentration of dissolved oxygen.

26. The method according to claim 24, further comprising:
determining a dissolved oxygen content of the mixture during the yeast propagation;
adjusting an aeration of the mixture during the yeast propagation on the basis of the deviation between the actual value and the reference value of the extract content and the dissolved oxygen content.

27. The method according to claim 16, further comprising:
determining a pitching concentration of the yeast at the start time.

28. The method according to claim 27, further comprising:
determining a biomass concentration of the yeast on the basis of the propagation model, the pitching concentration, and a target value for the yeast concentration that is related to the target value for the residual extract content.

29. The method according to claim 28,
determining a viable yeast cell concentration during the propagation on the basis of the biomass concentration.

30. The method according to claim 16, further comprising:
determining a pH value of the mixture.
